(19) United States
(12) Patent Application Publication
(10) Pub. No.: US 2013/0211277 A1
(43) Pub. Date: Aug. 15, 2013

(54) System And Method For Reducing Stress Levels Through A Biofeedback Mediated System Using Color, Sound, And Aroma Therapy

(71) Applicants: Oyvind Berg, Boca Raton, FL (US); Elisabeth Rosse, Bordeaux (FR); Jean-Michel Bascole, Martillac (FR)

(72) Inventors: Oyvind Berg, Boca Raton, FL (US); Elisabeth Rosse, Bordeaux (FR); Jean-Michel Bascole, Martillac (FR)

(21) Appl. No.: 13/370,524

(22) Filed: Feb. 10, 2012

(51) Int. Cl. A61M 21/02; A61B 5/053

(52) U.S. Cl. 600/547; 600/27; 600/28

(57) ABSTRACT

The present invention is directed toward a system and method utilizing a biofeedback mediated color, sound, and aroma therapy. The visual, auditory, and aromatic stimulation is intended to alleviate stress and/or anxiety by relaxing and stimulating the brain, thereby correcting any neurophysical imbalances. Reduction in the levels of stress and/or anxiety is achieved through the use of the auditory, visual, and aromatic sequences which are individually specific and systematic. The system and method is preferably provided in multiple sessions with each session providing both a mechanism for providing treatment as well as assessment which is used to form the basis for future treatments.

### FIELD OF THE INVENTION

The present invention relates generally to a system and method that reduces stress, anxiety, and stress related disorders; and more particularly, to a non-pharmacological system and method which reduces the levels of stress and provides a mechanism for relaxation through a biofeedback mediated system and method which utilizes color, sound, and aroma therapy.

### BACKGROUND OF THE INVENTION

Technological advances over the last 50 years have provided individuals with the tools to accomplish tasks more efficiently. While such advances have provided the necessary means to conduct business or personal tasks faster and easier, such innovation has resulted in a complex and fast-paced society. For example, smart phone technology has been developed to allow users the ability to be technologically connected to clients, co-workers, or employers when not physically at their work site. While such technology makes life simpler in many respects, the fact that the user is continuously “wired’ or connected adds a certain amount of stress, as there is no down time in which the individual does not have to answer to the demands of clients, coworkers, or bosses.

Stress is usually defined as the “feeling” a person gets when overwhelmed or is undergoing life changing events. In fact, any type of event, either big or small, that may pose a threat to an individual's well being acts as a stressor and results in a stress response. Stress response is the body's mechanism for handling some type of trigger event, thereby allowing the body to respond. The stress response allows the individual to accomplish tasks efficiently and provides the individual the ability to react in dangerous situations in which the reaction must be automatic and swift. Accordingly, when a stress event occurs, there are definite physiological reactions that take place in the body. For example, stress situations often produce flight or fight stress reaction chemicals, such as cortisol, adrenaline, and noradrenalin. The production of these chemicals results in changes in blood pressure and heart rate, changes in breathing, and changes in muscular functioning. While changes in these systems are beneficial in the short term, long term stresses can result in pathological conditions that are harmful. Additionally, the inability to handle even short term stress can result in damage to one's physical and mental health.

Since individuals have different coping skills, treating stress has been accomplished in a variety of manners. In severe stress related diseases, and for individuals who can not find other relief means, pharmacological agents are used. These medications, however, are not always effective and can result in developing a drug dependency. Accordingly, a non-pharmacological method for reducing stress is needed.

### SUMMARY OF THE INVENTION

A system and method utilizing a biofeedback mediated color, sound, and aroma therapy is described. The visual, auditory, and aromatic stimulation is intended to alleviate stress and/or anxiety by relaxing and stimulating the brain, thereby correcting any neurophysical imbalances. Reduction in the levels of stress and/or anxiety is achieved through use of the auditory, visual, and aromatic sequences which are individually specific and systematic. The system and method are preferably provided in multiple sessions with each session providing both a mechanism for providing treatment, as well as user assessment which forms the basis for future treatments.

The system and method combine colors, shapes, sounds, images and aroma, along with biomedical electronics, to de-stress the mind and body. This unique concept enables the conscious and the unconscious intelligence of the brain to react differently to stressors, thus providing a state of balance and equilibrium. Such actions are partially based on the principles of chromotherapy, music therapy, and aromatherapy.

Chromo therapy is the science that uses color to preserve and restore the body's vibrational frequencies that correspond to physical and mental wellness. It is a natural method based on the fundamental properties of each color having strength, action and temperature that characterizes it and initiates reactions inside the body. Restorative effects can be obtained by using the color corresponding to the element lacking in the body or the color with the exceeding essence in the body. When balanced, optimal health is maintained or regained.

Music therapy uses the psychological, affective and relational effects of music: noises, voices, sounds, silence, breath and movement. Sound is a mechanical compression wave that propagates through a solid, liquid, or gaseous environment characterized by vibration, amplitude and tone. Music has the ability to manipulate and make us feel various and strong emotions.

The system and method in accordance with the present invention uses not only color and music, but also moving shapes and images as well as aromas to enhance the therapeutic value of the experience. Aromatherapy is also filled with vibrations of an electromagnetic spectrum. Such therapy helps promote the healing of the body as well as activating the brain's emotional centers for peacefulness of the mind. Such a system and method is adapted to create the perfect blend of color, music and aroma for an optimal session for each client.

To obtain balance, wellness and daily efficiency, the present invention creates a unique diagnostic system to create each personalized session, using four steps: Step 1 color reacting test (Color Tester); Step 2 personalized questionnaire; Step 3 biofeedback; and Step 4 session of senses harmonized. In the first part of the session, the user is immersed in a bath of personalized color preparing them for harmonization. The user is then taken into the heart of their prevailing element and exposed to theme-based images, such as but not limited to, water (life awakening and perennial), air (imagination and symbol of freedom); earth (symbol of wisdom and broad-mindedness); fire (symbol of life strength, brightness and power).

During each session, the system will choose a movie based on the four elements using symbolic images of nature and geometry. To complete the treatment, the last the patient recovers their unity with meditation supported by a viewing images of a mandala (geometric design symbolic of the universe) and corresponding color which enhances creativity and the ability to advance more positively toward the future. Accordingly, each session is based on four components: the association of moving shapes (concentric circles based on hypnotherapy); infusion of aromatherapy; color, sounds and music with images involving the theme-based images, and calming images such as the appearance of mandala, each corresponding with a restoring and curing color. The present invention also includes a computer based system to record all the information from one session to the next and follows the evolution of the client to adjust the parameters in order to determine and optimize results, such as the types of images viewed and the color and aroma association of the images.

An illustrative embodiment of the non-pharmacological method of reducing anxiety and stress levels within an individual in accordance with the present invention may comprise the steps of: assessing an individual's psychological and physiological status by subjecting said individual to one or more first set of tests; electronically analyzing said data to obtain a first treatment session designed to reduce stress levels; providing said first treatment session to said individual, said treatment session having a combination of visual, auditory, and aromatherapy; and providing additional treatment sessions, each said additional treatment session comprising the steps of assessing said individual's psychological and physiological status by subjecting said individual to one or more first set of tests, providing a second treatment session to said individual, said second treatment session having a combination visual, auditory, and aromatherapy; assessing said individual's physiological status by subjecting said individual to one or more second set of tests; obtaining data for each said test, and electronically analyzing said data to obtain the next treatment session based on the indication of the user's stress-related profile obtained by the data analysis.

Accordingly, it is an objective of the present invention to provide a system and method for reduction of stress and/or anxiety.

It is a further objective of the present invention to provide an interactive stress management system and method designed to provide deep and lasting relaxation through stabilization and balancing of the central nervous system.

It is yet another objective of the present invention to provide an interactive stress management system and method designed to provide deep and lasting relaxation through stabilization and balancing of the central nervous system which uses a combination of color therapy, sound therapy, and aroma therapy.

It is a still further objective of the present invention to provide an interactive stress management system and method designed to provide deep and lasting relaxation through stabilization and balancing of the central nervous system which uses a combination of color therapy, sound therapy, and aroma therapy, as well as physiological analysis of the individual undergoing the treatment.

It is a further objective of the present invention to provide an interactive stress management system and method designed to provide deep and lasting relaxation through stabilization and balancing of the central nervous system which uses a combination of color therapy, sound therapy, and aroma therapy, as well as psychological analysis of the individual undergoing the treatment.

It is yet another objective of the present invention to provide an interactive stress management system and method designed to provide deep and lasting relaxation through stabilization and balancing of the central nervous system which uses a combination of color therapy, sound therapy, aroma therapy, as well as physiological and psychological analysis of the individual undergoing the treatment.

It is a still further objective of the present invention to provide an interactive stress management system and method designed to analyze the status of the patient in one session in order to provide a treatment for a following session.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a schematic diagram of the hardware components of the stress and anxiety reducing biofeedback system in accordance with one embodiment of the present invention;

FIG. 2 illustrates an embodiment of the anxiety and stress reducing biofeedback system in which the visual, audible, and aromatic components are assembled as a single unit;

FIG. 3 illustrates an embodiment of the anxiety and stress reducing biofeedback system in which the visual, audible, and aromatic components are assembled as independent components;

FIG. 4 is a block diagram of the components of an illustrative embodiment of the morphological hand adaptive system in accordance with the present invention;

FIG. 5 is a perspective view of an alternative embodiment of the morphological hand adaptive system;

FIG. 6 is a block diagram of the components of an illustrative embodiment of the bio-impedance modality in accordance with the present invention;

FIG. 7 illustrates an embodiment of the bio-impedance testing, showing use of an electrode placed on a user's hand in order to measure changes related to the bio-impedance testing;

FIG. 8 is a block diagram of the components of an illustrative embodiment of the hydration modality in accordance with the present invention;

FIG. 9A is an illustrative example of images of the geometrical shapes, such as a colored spiral, associated with the treatment videos;

FIG. 9B is an illustrative example of images of the geometrical shapes, such as colored triangles, associated with the treatment videos;

FIG. 9C is an illustrative example of images of the geometrical shapes, such as colored waves, associated with the treatment videos;

FIG. 10A is an illustrative example of a theme-based image, such as a volcano eruption, associated with the treatment videos;

FIG. 10B is an illustrative example of a theme-based image, such as clouds in the sky, associated with the treatment videos;

FIG. 10C is an illustrative example of a theme-based image, such as a water fall, associated with the treatment videos;

FIG. 11 is a flow chart describing an illustrative example of the steps of the method of reducing anxiety and stress in accordance with the present invention.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

The present invention incorporates and improves upon the subject matter of a previous biofeedback system developed by Harmonial Corporation. (Boca Raton, Fla., USA). The previous system was based on a combination of color and sound therapy. The current system combines color and sound therapy with aromatherapy to provide an enhanced biofeedback mediated therapy designed to reduce stress and/or anxiety, and to treat symptoms of individuals suffering from the effects of stress and anxiety. The system and method further incorporates real-time and continuous evaluation methods to assess the physiological and psychological status of the user as the treatment is being undertaken. The evaluation methods provide data which is electronically analyzed by a computer having specialized software programs which establish future treatment sessions, i.e. the appearance of specific shapes and images, arrangement and synchronization of the colors, sounds and aromatherapies.

The non-pharmacological method of reducing anxiety and stress levels within an individual as described herein provides sessions for which color treatment has been personalized based on reactions to various testing methods. The action of the method is chromatic, sonorous, and aromatic. The personalized color treatments are designed to penetrate through the eyes and regulate the biological clock of the body through action on the hypothalamus. The hypothalamus controls the nervous system and the endocrinal system, which together regulate all the biological functions of the human body. It sends information linked to light to the pineal gland, which informs other organs of it. The cells of the pineal gland, in turn, transform the nerve impulses resulting in conversion of luminous messages into chemical molecules.

The methodology in accordance with the present invention is used as a compliment to classical medical treatments, homeopathy, or acupuncture in order to improve the general state of the user. For any person, there exists a permanent equilibrium between the body and the psyche. Both physical and psychological negative experiences can create states of ill-being. Anxiety or depression, for example, create imbalances in a person's cerebral hemispheres which can result in repercussions in our daily behavior, such as excessive tiredness or trouble sleeping. Treatments using color are designed to rebalance individuals suffering from imbalances resulting from physical pathologies brought about by psychological troubles, anxiety, depression, and/or troubles adapting to stress.

The methodology in accordance with the present invention further includes use of sound treatments in combination with color treatments. Use of color and sound, therefore, are designed to provide enhanced rebalancing mechanisms. Sound is a vibration which penetrates the skin and bathes our body entirely. Different sound frequencies contact our cells and our molecules in different ways and help them to adjust and re-harmonize. Sickness, physical, emotional or mental problems indicate an incorrect vibratory frequency or an energy blockage between these different levels of existence. Each color and each sound possess a vibratory frequency and harmonic interval capable of re-establishing or re-training the sick body, emotions and thoughts.

Sound is similar to color, differing by a lesser vibratory frequency. In addition, sound also has harmonic properties that color does not have. Harmonics (the sharps above fundamental sound) have the capacity to stimulate the brain (epiphysis) and to produce states of modified consciousness facilitating relaxation (alpha waves). Sound recharges the cortex and drives the nervous system. Sound travels and stimulates different zones of the brain and can balance the 2 hemispheres. Moreover, sonorous vibrations impact the body through resonance with the cellular fluids in the body. Vibrations alternating high and low frequencies as in the sessions of the present methodology are designed to improve functions of the mechanism of the ear. Sound allows access to emotions buried in the unconscious and favors the releasing of “emotional crystals” with the spontaneous appearance of images of the past accompanied sometimes by sadness and anger not yet transformed. Additionally, sound brings about relief through its effect on the central nervous system through improving cognitive capabilities (attention, memory) and psycho-motor capabilities through which it is known to reduce the effects of stress. The effectiveness of the methodology in accordance with the present invention is made possible by the combination of several Ayurvedic and Chinese fields of knowledge and by the association of several techniques stimulating the sensory apparatus by the bias of hearing, sight and smell. It is this vibratory synthesis uniting waves of sound, color, and smell which act on the totality of the cerebral and corporeal functioning of the patient in order to reestablish harmonious balance.

The methodology in accordance with the present invention is designed to have an effect on the brain. As a result of cerebral plasticity, the brain modifies the organization of its networks of neurons under the influence of sounds, colors and smells, which have an immediate effect on the cerebral treatment of emotion. The two hemispheres of the brain play a role. The left hemisphere is in control of rhythm and form, while the right hemisphere controls melody, harmony, and color. The cerebral structures involved are found in the right medio-temporal lobe, where they are stimulated. The hippocampus is important for the functioning of memory and the fine analysis of harmony. The amygdala region is important in regulation of emotions. The hypothalamus is essential for the regulating of biological rhythms. It has been shown that sound has an influence on cerebral activity, through the effect of resonance within the bony, muscular, tissue, or nerve structures of an individual, influencing the pulse, breathing and emotions, lowering arterial tension, cardiac rhythm, muscular tension, which reverberates on the spirits and anxiety. The impact of the methodology in accordance with the present invention contributes to re-harmonizing the electromagnetism of the brain. The visualization of colors, as well as the combined action of sounds and smells spread sonorous, chromatic and olfactory rebalancing vibrations through the skin and mucus, causing internal regulation in the vibratory behavior of the cells. In addition, the method works as a result of the symbolism of the images and forms used.

In general, the system works using several components, an evaluation, development of the treatment of a session based on the evaluation, and debriefing. The evaluation uses a plurality of testing modalities. A questionnaire is used to define the general profile of the patient. A color test is used to evaluate the characteristics of the muscular power of each pressure of the patient in response to the colors previewed, allowing a determination of the patient's luminous frequency of rebalancing. A hydration test is used to provide indications on the positive or negative state of emotion important to the patient. A measure of bio-impedance used throughout the session provides the state of the nervous and muscular tension of the patient. From the analysis of the questionnaire, in conjunction with the results of the color test, bio-impedance test, and hydration measurement, an energy profile of the patient will be determined, allowing the establishment of personalized sessions during which one color and its complement, images relating to theme based criteria, such as earth, water, fire or air representing symbolically the emotional state of the patient are used, and additional images, such as mandala (circle) and a rainbow will be utilized. One or more scents will be used in conjunction with the color/sound/image treatments.

As an illustrative example, if the results of the evaluation demonstrate an excess of humidity objectified by the hydration test and cross checked with the responses to the questionnaire, the color green, whose frequency will spur the body to produce a drying reply will be used, and the element theme based images of water representing the current state of the patient will be proposed. In fact, each color sends to the body a “code” message in the form of electro-magnetic radiation of a determined length of waves and each frequency generates a unique effect. Each element symbolically represents an interior state, which, if it is put into images may help to change the emotional state of the patient. Such emotional state can be associated them, among others, four qualities, hot, cold, dryness and humidity. The method in accordance with the present invention, through the visualization of colors of wave form, in combination with the action of symbols, sounds and smells spreads through the sensory apparatus, the skin and mucus, sonorous, olfactory vibrations (aromatic molecules are caught by thousands of chemo-receptors in the olfactory epithelium to be transmitted to the olfaction centre, in the heart of the limbic brain, which controls our neuro-vegetative system and all our psychological states, provoking psychic effects, reactions independent of will, which may allow the modification of behaviors with regard to addictions: bulimia, nicotinism, alcoholism) leading to an internal regulation in the vibratory behavior of cells.

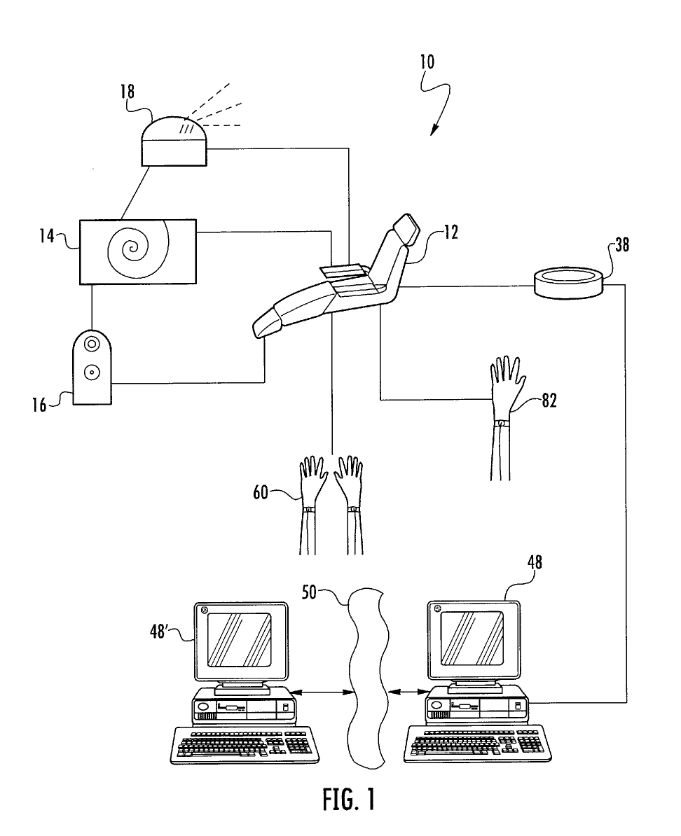

Referring to FIG. 1, a schematic diagram of the hardware components of the anxiety and stress reducing biofeedback system 10 in accordance with an exemplary embodiment of the present invention is illustrated. The anxiety and stress reducing biofeedback system 10 includes a patient resting zone 12. The patient resting zone may include a chair, couch, arm chair, or dental-style chair, in which the individual seeking treatment may sit and/or lay comfortably while viewing or receiving one or more visual, audible, and aromatic stimulating programs. One or more display units 14, such as a flat screen LED television, a plasma television, television monitor, or a HDTV projection unit, is placed within a viewable distance from the patient resting zone 12. One or more electromechanical devices for producing sound 16, such as a speaker, are used to provide the user with a variety of auditory experiences. An aroma dispensing device 18 is utilized to provide a mechanism that dispenses volatile plant materials, essential oils, or aromatic compounds to the user. The resting zone 12, the one or more display units 14, the electromechanical devices for producing sound 16, and the aroma dispensing device 18 can be designed as a single unit. In an alternative embodiment, one or more of the components may be placed in goggles and/or within a helmet.

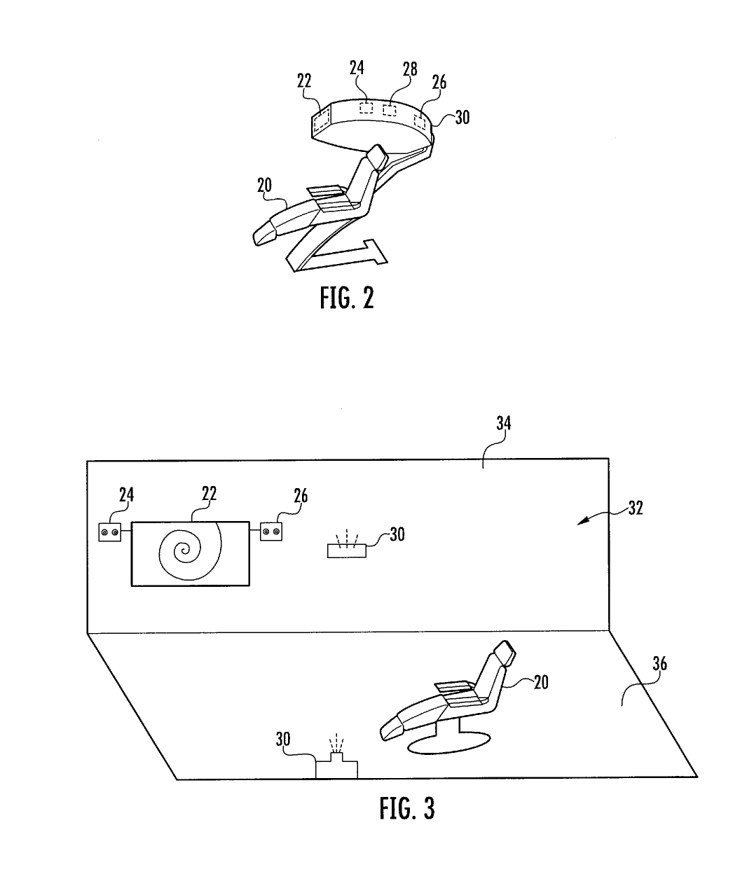

FIG. 2 illustrates a single unit embodiment in which the patient resting zone 12, illustrated herein as a reclineable dental-style chair 20 includes a LCD television monitor 22, a plurality of speakers 24 and 26, and an aroma dispensing unit 28 integrated into unit 30. The unit 30 is attached to the reclineable dental-style chair 20. Alternatively, the anxiety and stress reducing biofeedback system 10 includes the reclineable dental-style chair 20, the LCD television monitor 22, the plurality of speakers 24 and 26, and the aroma dispensing unit 28 as separate units arranged in a predefined space, such as an enclosed room 32, and placed on walls 34 and/or the floor 36 of the enclosed room 32, see FIG. 3.

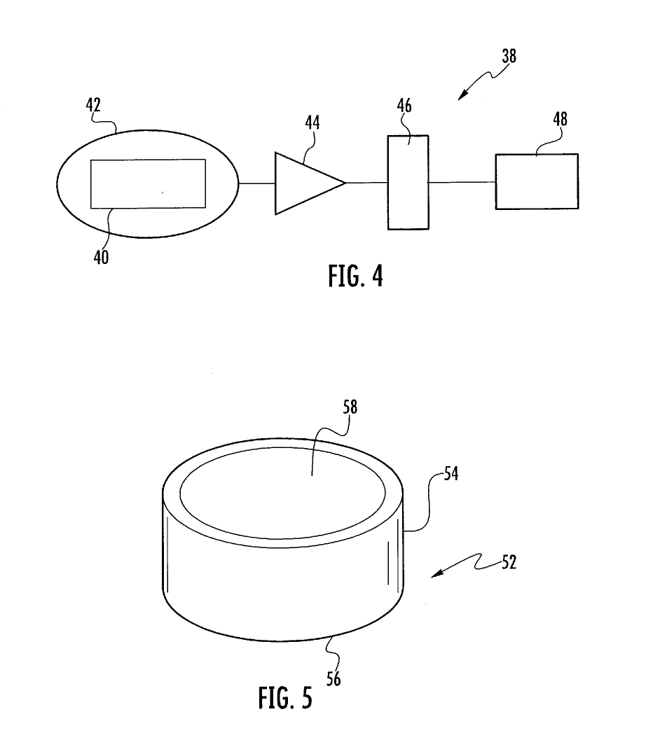

Referring back to FIG. 1, the anxiety and stress reducing biofeedback system 10 further includes one or more physiological/psychological detection modules to provide data which can be analyzed, processed and correlated to provide future treatment sessions. For example, the anxiety and stress reducing biofeedback system 10 may include a color tester illustrated herein as a morphological hand adaptive system 38. Referring to FIG. 4, a block diagram of an illustrative example of the morphological hand adaptive system 38 is shown and includes a sensor 40 for detecting a pressure applied to the sensor 40. The sensor 40 is enclosed by an outer surface 42, such as a rubber ball, which is designed to be held in the hand of the user. The sensor 40 is designed to detect changes in the diameter of the ball in response to pressure applied thereto. Changes in the diameter can then be converted into a precise analysis of the changes in pressure caused by the user's hand contacting the ball and applying different amounts of pressure to the rubber ball in response to visual stimulation.

The sensor 40 is electrically coupled to an amplifier 44, such as an operational amplifier. The amplifier 44 is designed to enhance the signal of the sensor 40 and/or modify the signal level to be compatible with a microprocessor 46. The modification of the signal level may be, for example, converting the signal from an analog signal to a digital signal. Additionally, the sensor 40 is also designed to continuously obtain pressure data information. The real-time, continuous data acquisition regarding pressure is stored in the microprocessor 46 and allows for very precise measurements for analysis in changes in hand pressure. Moreover, the real-time data acquisition allows the anxiety and stress reducing biofeedback system 10 to combine pressure and reaction time as part of the overall analysis. All data obtained is then transferred to a main computer 48. The main computer 48 preferably contains one or more of: Central Processing Unit (CPU), memory, storage, input control, modem, network interface, and the necessary software specifically designed to provide analysis and determinations of treatment options. Additionally, one or more remotely located computer 48′ can be used for storage and/or data analysis. Remote computer 48′ is accessible through the internet 50.

FIG. 5 illustrates an alternative embodiment to the morphological hand adaptive system shaped in the form of a rubber ball. The alternative embodiment to morphological hand adaptive system includes a hockey puck shaped block main body 52. The main body 52 contains a first portion 54 and a bottom portion 56. Placed on the first portion is a foam member 58 which is coupled to a sensor. The foam member 58 is adapted to be squeezed by the entire hand or individual fingers. The sensor senses pressure applied to the foam member 58 and relays the information to a microprocessor for analysis and storage.

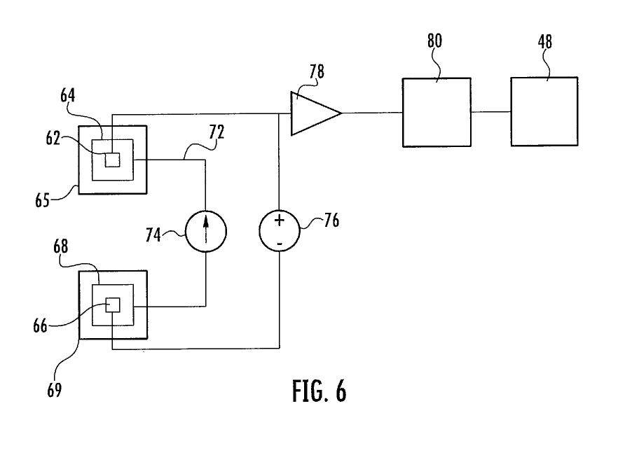

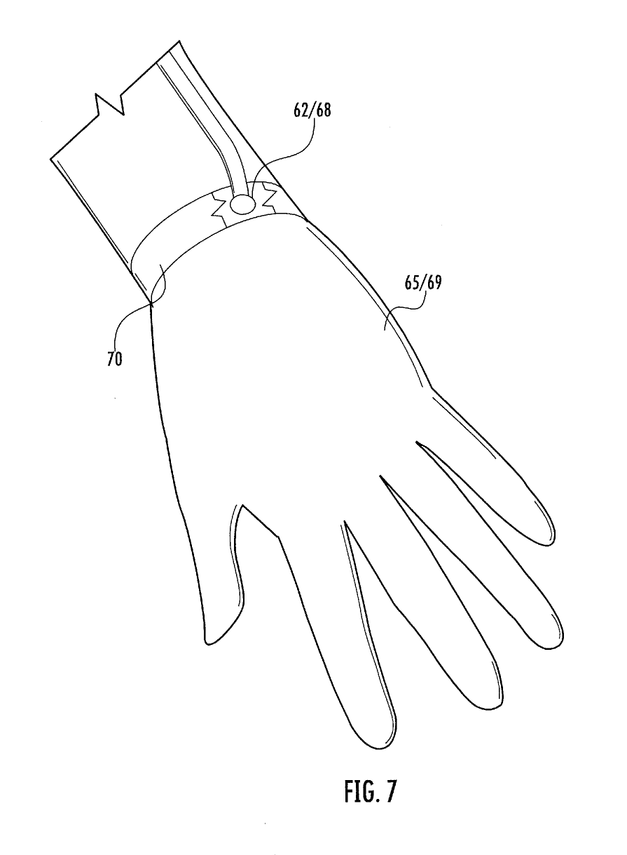

The anxiety and stress reducing biofeedback system 10 also includes additional physiological/psychological detection modules. Referring to FIG. 6, a block diagram of a bio-impedance analysis module 60 is illustrated. The bio-impedance analysis module 60 is based on the principal of determining opposition of the flow of electrical current through body tissue. A first electrode 62 and a second electrode 64 are attached to the left hand 65 of the individual. A third electrode 66 and a fourth electrode 68 are attached to the right hand 69 of the individual. Electrodes may be attached to the user's hands, 65 and 69 through use of a strap 70, see FIG. 7. A current 72 is generated by a generator 74, such as a Howland generator. The measured voltage 76 is derived from the current source generator 74. The measured voltage 76 is proportional to the body impedance. A converter 78 converts the bio-impedance data from an analog signal to a digital signal. A microprocessor 80 processes the data and stores the data in computer 48 and/or sends the data to secondary, remote computers 48′.

Several of the tests used in the evaluation process rely on the measurement of voltage. Such change in voltage may be a result of physiological changes, such as muscle tension or blood tension. Based on how the body is reacting to visualization of the videos/sounds/aroma, change in voltage is indicated by muscle tension or blood pressure. The change in voltage is correlated into selection of available libraries of selected videos and graphics which form the basis of present and future treatment sessions. Such video libraries contain sessions that synchronize color treatments with music and aromas.

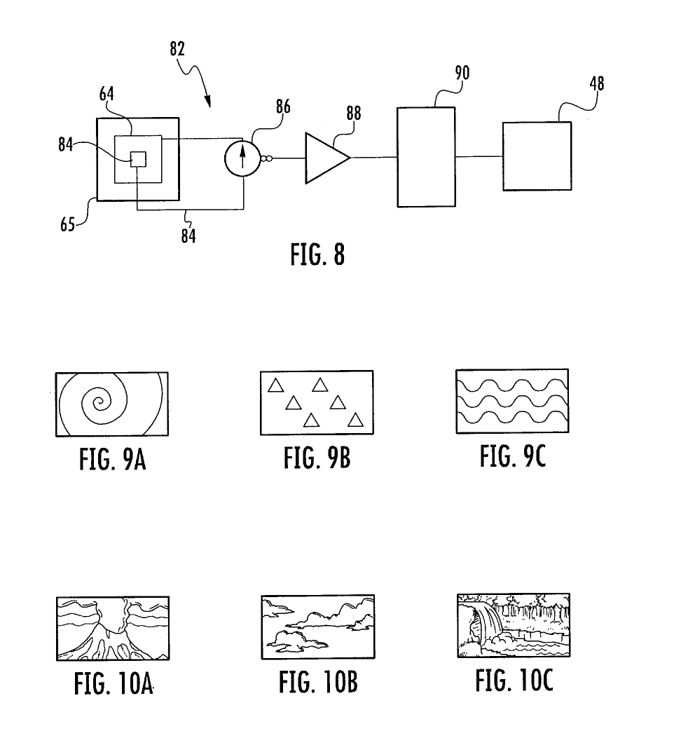

A fourth measurement modality in accordance with the present invention includes a hydration measurement modality 82, see FIG. 8. The hydration measurement modality 82 includes an electrode 84 which is attached to the hand, preferably the right hand 65, of the user. A current 85 is generated by a generator 86 between electrodes 64 and 86 and applied to the skin of the user's hand 65. Voltage readings are extracted from the generator to obtain an image of the local impedance of the skin. Similar to the bio-impedance analysis module 60, a converter 88 converts the hydration data from an analog signal to a digital signal. A microprocessor 90 processes the data and stores the data in computer 48 and/or sends the data to one or more secondary, remote computers 48′. The microprocessors or computers used within the system may contain one or more algorithms stored within a memory for correlating data from the analysis portions (questionnaire, color tester, bio-impedance and hydration testing) to create a treatment session, such as a video having a particular sequence of color, sound, aroma, and visual image exposure to the user.

The anxiety and stress reducing biofeedback system 10 is an interactive stress management system which is designed to provide a method for deep and lasting relaxation through stabilization and balancing of the central nervous system. The method in accordance with the present invention is unique in that it is designed to reduce stress while providing a determination of the individual's receptivity to color, sound, and aroma in order to characterize treatment effectiveness to provide future color, sound, and aroma treatment sessions.

In use, the anxiety and stress reducing biofeedback system 10 provides individualized, progressive treatments using color, music, and aromatherapy which are unique to each user, and based on data generated as the user undergoes one or more treatment sessions. The auditory, visual, and aromatic stimulation is designed to alleviate and correct any neurophysiologic imbalances. The method includes exposing users to videos designed to incorporate color, visually themed images and music in combination with aromatherapy. The method incorporates analysis mechanisms both prior to and concurrently with each video treatment.

The method of treatment in accordance with the present invention is performed in a series of progressive sessions. Each session is designed to provide a program, i.e. video session, which combines color therapy, music therapy, and aromatherapy to reduce the levels of stress and/or anxiety of the user. One of the main components of the treatment is the use of colors to preserve or restore the body frequency corresponding to physical and mental health. Reduction of stress and anxiety can be accomplished by using color-based therapy and/or by providing color based treatment options based on color-associated imbalances. The color treatment includes both exposure to colored light, colored images, and theme-based images associated with a particular color, see Table 1. Additionally, each color treatment is matched with specific musical notes or arrangements, see Table 2, as well as specific essential oils or aromas, see Table 3. The combination of the color treatment, music treatment, and aromatherapy was found to provide enhanced reduction in stress and/or anxiety.

TABLE 1
Association of Theme-based Images with Color Treatment:

| Color | Theme Based/Element Therapy |
|---|---|
| Red | Images Relating to Fire |
| Orange, Yellow, White | Images Relating to Earth |
| Blue, Indigo, Violet | Images Relating to Air |
| Green | Images Relating to Water |

TABLE 2
Music Therapy: Association of Color Treatment
with Music Treatment

| Color | Music |
|---|---|
| Red | Do/C |
| Orange | Re/D |
| Yellow | Mi/E |
| Green | Fa/F |
| Blue | Sol/G |
| Indigo | La/A |
| Purple | Si/B |
| White | Si/B |

TABLE 3
Aroma Therapy: Association of Color Treatment

| Color | Fragrance/Essential Oil |
|---|---|
| Red | Carnation/Rose |
| Orange | Orange Flavor |
| Yellow | Bergamot |
| Green | Mint |
| Blue | Thyme |
| Indigo | Jasmine |
| Purple | Lavender |
| White | Cajuput/Myrtle |

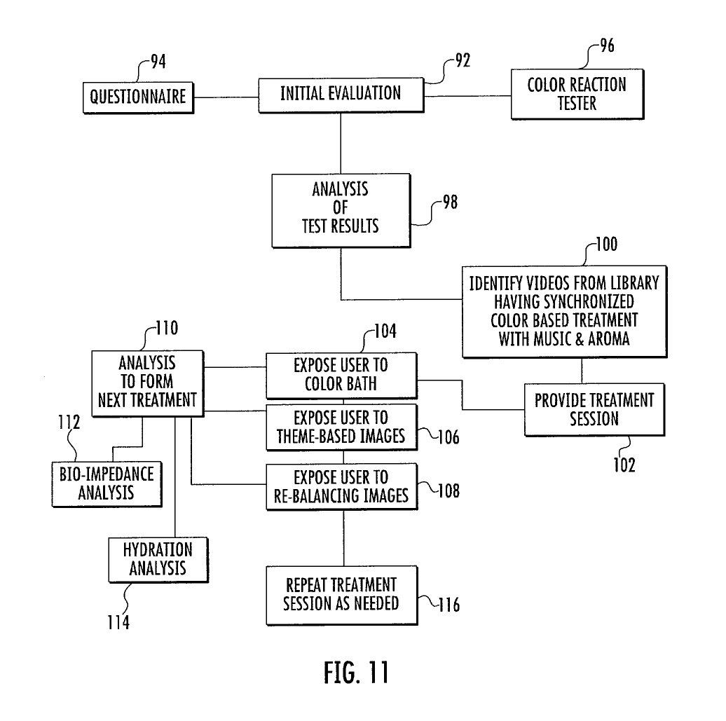

FIG. 11 outlines the steps undertaken for the methodology. The method consists of a plurality of analysis modules: 1) a questionnaire, 2) a color analysis tester (pressure/time reaction test), 3) a bio-impedance test, and 4) a hydration test, as well as treatment sessions. In an illustrative example, the system utilizes one or more algorithms stored within a memory of the microprocessors or computers for correlating data from the analysis portions (questionnaire, color tester, bio-impedance and hydration testing) to create a treatment session, such as a video having a particular sequence of color, sound, aroma, and visual image exposure to the user. In use, an individual desiring relaxation undergoes the first of multiple sessions. After a first initial assessment, each corresponding treatment is designed to correct any neuropathological imbalances through the synchronization of color, sound and aroma therapy. The initial session (92) includes an evaluation of the individual in order to provide the individualized, specific color/music/aromatherapy treatment. Preferably, but not necessary, the individual is placed in a quiet, secluded area and may or may not be resting on the patient resting zone 12, see FIG. 2. The individual is provided a questionnaire (94) having a plurality of questions designed to determine a treatment option based on their status, i.e. imbalance, state of stress. The questionnaire may contain any number of questions specifically designed to provide an assessment of the user. For example, an illustrative embodiment of a questionnaire may contain forty two questions with each question having a two answer possibility, affirmative or negative. The questionnaire is designed so that each color category has a percentage of questions which if answered in a certain manner leads to that color treatment being determined as the treatment color, TC.

| | | PREFERRED |
|---|---|---|
| QUESTION | RESPONSE | RESPONSE |
| 1. Currently, do you have a cold? | Yes/No | Yes |
| 2. Have you had an emotional or | Yes/No | Yes |
| physical trauma recently? | | |
| 3. Do you have circulatory disorders? | Yes/No | Yes |
| 4. Do you have a good appetite? | Yes/No | No |
| 5. Are you cold sensitive? | Yes/No | Yes |
| 6. Are you rather hesitant when you | Yes/No | Yes |
| must act? | | |

The six illustrative questions are related to the TC of red, so that depending on the answers to these questions, the TC red would be chosen or eliminated. For example, if the user identified four out of the six questions according to the preferred response, the TC red was assigned to that person for that treatment option. After completing the questionnaire, the answers are analyzed by a software system which is programmed to determine the TC based on the answers provided.

Once the questionnaire has been undertaken, the individual is subjected to a second treatment determining test, the color test (96). The color test is designed to determine the user's reaction to colors visually displayed on a screen or display unit. A color testing device having a sensor for determining a pressure exerted upon the device, as well as reaction time of the user in applying the pressure, such as described previously, i.e. the color ball or the tester depicted in FIG. 5, is provided to the user. The color test begins by showing the individual a series of colors, i.e. red, blue, green, on a screen or display unit. The series of colors may be arranged in a specific manner, i.e. from warm colors to dark colors or dark colors to warm colors, or may be shown randomly. The individual holds or places one or both hands on top of one (or two) color testing device. As the colors are presented to the individual, the individual interacts, i.e. squeezes the ball, with the sensing device.

The test is designed to measure the user's reaction to each color by measuring the pressure exerted and the user's reaction time. The pressure and reaction time for each color differs and such differences are used in the overall evaluation process. The data is stored to a computer system and analyzed by software which determines a TC value. The test measures the quality of muscle reflex, and that measure is then interpreted to provide information as to how the body reacts to the received color. This determination is used to provide further treatment strategies and options. Determination of the TC through information obtained by the questionnaire and the color tester constitutes the first session.

The TC determined above provides the user with an individualized treatment session. The treatment session comprises a combination color, music, and aromatherapy designed to impact the brain and provide deep and lasting relaxation through stabilization of the central nervous system. Analysis of the test results (98) is used to identify videos from a video library (100) containing variations of video scenes that are correlated to the data received from the analysis steps of the method. Such videos are used as the treatment session (102). Each video is specifically designed to be used based on the condition of the individual as determined by initial testing and previous treatment session. Preferably, the treatment session lasts between 10 to 26 minutes, but can be designed to be of longer or shorter duration. The treatment preferably contains three parts. The first part includes a color bath (104) video shown to the individual, preferably lasting 4-7 minutes. This portion of the video contains color exposure and geometrically shaped images that move upon a screen or display unit, see FIGS. 9A-9C. The video further contains music which contains one or more specially arranged pitches, frequencies, and arrangements to coincide with the movement of the colors and/or geometrically shaped images. The treatment session further provides for a 5-10 minute video which subjects the user to images based on a particularized theme (106). The theme-based video may be shown to the user as a continuation of the color bath video, or may be a separate independent video. The particular theme is arranged to provide images, colors, or combinations thereof, which accomplish body harmonization and stability. In contrast to the video which displays geometrical shapes, this portion includes images of objects, such as but not limited to water and bubbles or a gong, or particular scenery, settings, or landscapes, see for example FIGS. 10A-10C. As an illustrative example, the theme-based video session may focus on providing images based on one or a combination of 1) earth, such as images of forest, trees, 2) fire images that connote fire or scenes, such as images of flames, volcano eruption, fire burning scenes or fireplaces, 3) water, such as images of waves, ponds, streams, boiling water, water falls, and 4) air such as the sky, cloud formation. The treatment session further contains a third video portion (108) which includes providing images which result in a calming effect, preferably images that aid in meditation or trance induction. An illustrative example may include one or more images of a Mandela and/or a rainbow.

During the entire session, or intermittently throughout the session, the user not only is visualizing the video, but is also being subjected to one or more aromas diffused directly to the user or within the treatment area. The one or more aromas that the user is being subjected to is specifically designed to work in combination with the particular videos, i.e. images, colors, and music, to effect the user in a particular manner. Referring to Table 3, the aroma that the user is exposed to corresponds to the particular color treatment. For example, should the TC be determined to be red, a carnation or rose fragrance will be used. Similarly, if the TC is determined to be orange, an orange flavored fragrance will be used as the aromatherapy. While Table 3 illustrates fragrances including carnation/rose, orange flower, bergamot, mint, thyme, jasmine, lavender, or cajuput/myrtle, other fragrances may be used and the list is not intended to be limiting.

Attached to the individual undergoing the treatments are one or more sensors designed to obtain physiological and/or psychological data (110) about the individual as he/she is partaking in the video session. The sensors are designed to provide real time acquisition of data relating to how the user is responding to the treatment. The data obtained is used to provide future treatments. As described previously, a bio-impedance testing (112) is performed by placing two electrodes onto the left and right hand of the user. A current is provided, and voltage, which is proportional to the body impedance, is measured. The data obtained is used to correlate bio-impedance with emotional reactions, and using software programming provides a TC based on such calculations. The change in impedance is used in the assignment of a treatment color. For example, increased impedance may be assigned a color red, while a decrease in impedance is assigned a color orange. The bio-impedance testing is utilized pre-video session, throughout the video session, as well as post video viewing, allowing real-time and continuous data acquisition. Such data acquisition allows for better determination of the user status and body reaction during the sessions, as well as more accurate TC determinations.

In addition to the bio-impedance testing, an electrode is placed on the right hand of the user for hydration measurements (114). The hydration electrode may be a separate electrode or may be part of the bio-impedance electrode. Similar to the bio-impedance testing, a current is generated to the skin of the right hand in order to obtain data relating to the local impedance of the skin. The impedance of the skin is correlated to the hydration of the skin and such correlation is used to provide a TC. Hydration testing is tied to the emotional reaction. Response to the session results in a physical reaction on the skin as the user responds to the session. The hydration testing is performed pre-video session, throughout the video session, as well as post video viewing, allowing real-time and continuous data acquisition. Such data acquisition allows for better determination of the user status and body reaction during the sessions, as well as more accurate TC determinations.

Additional treatment sessions are undertaken by the individual (116). Each additional treatment is executed in the same manner. Accordingly, each session will contain a questionnaire, a color test, and video session, and physiological and psychological data acquisition and analysis. Based on how the user reacts to the treatments, a new TC is determined after each treatment session. The new TC forms the basis of the type of video session, and corresponding color therapy and corresponding images, music therapy, and aromatherapy the user will be exposed to.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A non-pharmacological method of reducing anxiety and stress levels within an individual comprising the steps of:
assessing an individual's psychological and physiological status by subjecting said individual to one or more first set of tests;
electronically analyzing said data to obtain a first treatment session designed to reduce stress levels;
providing said first treatment session to said individual, said treatment session having a combination of visual, auditory, and aromatherapy; and
providing additional treatment sessions, each said additional treatment session comprising the steps of assessing said individual's psychological and physiological status by subjecting said individual to one or more first set of tests, providing a second treatment session to said individual, said second treatment session having a combination visual, auditory, and aromatherapy; assessing said individual's physiological status by subjecting said individual to one or more second set of tests; obtaining data for each said test, and electronically analyzing said data to obtain the next treatment session.

2. The non-pharmacological method of reducing anxiety and stress levels within an individual according to claim 1 wherein said step of assessing an individual's psychological and physiological status by subjecting said individual to one or more first set of tests includes the administration of a questionnaire containing one or more pre-determined questions designed to assess the individual's emotional state and the administration of a color tester.

3. The non-pharmacological method of reducing anxiety and stress levels within an individual according to claim 1 wherein the step of assessing said individual's physiological status by subjecting said individual to one or more second set of tests includes the steps of administering a bio-impedance test and a hydration test.

4. The non-pharmacological method of reducing anxiety and stress levels within an individual according to claim 1 wherein the step of providing said first or second treatment session to said individual includes the step of subjecting said individual to a video session in combination with aromatherapy, said video session having a first component which subjects said individual to video having one or more color images, moving geometrical shapes and an auditory component, a second session subjecting said individual to one or more theme-based images, and a third session subjecting said individual to stress reducing images.

5. The non-pharmacological method of reducing anxiety and stress levels within an individual according to claim 3 wherein said bio-impedance test and said hydration test are performed prior to, during, and after said video session.

6. A non-pharmacological method of reducing anxiety and stress levels within an individual comprising the steps of:
performing an initial assessment of an individual to determine the psychological state of said individual;
obtaining data based on said initial assessment;
transmitting said data to a computer for data analysis;
electronically analyzing said initial assessment data;
performing a first physiological assessment of said individual to determine a first physiological state;
obtaining data on said first physiological assessment;
transmitting said first physiological assessment to said computer for data analysis;
assigning a first treatment session based on said data analysis of said initial assessment data and said first physiological assessment;
providing a first treatment session said analysis of said initial assessment data and said first physiological assessment to said individual, said first treatment session using color therapy, auditory therapy, and aromatherapy;
providing a second treatment session, said second treatment session comprising the steps of:
performing a second assessment of said individual to determine the psychological state of said individual;
obtaining data based on said second initial assessment;
transmitting said data to a computer for data analysis;
electronically analyzing said second assessment data, said electronic analysis generating a second treatment session;
performing a second physiological assessment to determine a second physiological state;
obtaining data on said second physiological assessment;
transmitting said second physiological assessment to said computer for data analysis;
subjecting said individual to color therapy, auditory therapy, and aromatherapy;
obtaining data relating to said individual's physiological response to said color therapy, auditory therapy, and aroma therapy;
analyzing said data relating to said individual's physiological response to said color therapy, auditory therapy, and aromatherapy; and
determining a third treatment session based on analysis of said second initial assessment, said second physiological assessment, and said individual's physiological response.

7. The non-pharmacological method of reducing anxiety and stress levels within an individual according to claim 6 wherein said treatment session further comprises subjecting said individual to one or more video sessions, said video sessions comprising images, shapes, theme based images, or combinations thereof.

8. The non-pharmacological method of reducing anxiety and stress levels within an individual according to claim 6 wherein said step of subjecting said individual to one or more video sessions further includes the steps of:
subjecting said user to a first video session having a combination of colored images, moving geometrical shaped shapes, and music;
subjecting said individual to a second video session containing theme-based images, and
subjecting said individual to a third video session having images which result in relaxation.

9. The non-pharmacological method of reducing anxiety and stress levels within an individual according to claim 6 wherein said first and second initial assessment comprises a questionnaire.

10. The non-pharmacological method of reducing anxiety and stress levels within an individual according to claim 6 wherein the step of performing a first or second physiological assessment further includes the steps of visually displaying one or more colors to said subject, obtaining data relating to the muscle sensibility and reaction time to said one or more colors displayed, electronically analyzing said data.

11. The non-pharmacological method of reducing anxiety and stress levels within an individual according to claim 10 wherein the step of performing a physiological assessment further includes measuring the bio-impedance between the left hand and the right hand of said user, analyzing said measurements, and electronically analyzing said data to form a treatment session.

12. The non-pharmacological method of reducing anxiety and stress levels within an individual according to claim 11 wherein said analysis is performed prior to, during, and after said video session.

13. The non-pharmacological method of reducing anxiety and stress levels within an individual according to claim 6 wherein the step of performing a physiological assessment includes measuring the bio-impedance of the skin of said individual, correlating said measurement to the hydration of said skin, and analyzing said data to form a treatment session.

14. The non-pharmacological method of reducing anxiety and stress levels within an individual according to claim 13 wherein said analysis is performed prior to, during, and after said video session.
